(19) United States
(12) Patent Application Publication
(10) Pub. No.: US 2025/0322945 A1
(43) Pub. Date: Oct. 16, 2025

(54) TRACKING AND ANALYZING INFORMATION IN INDIVIDUAL MEDICAL DEVICE TO IDENTIFY POTENTIAL ISSUES WITH THE INDIVIDUAL MEDICAL DEVICE AND TO UPDATE THE INDIVIDUAL MEDICAL DEVICE

(71) Applicant: Terumo BCT, Inc., Lakewood, CO (US)

(72) Inventor: John PITTINGER, Evergreen, CO (US)

(21) Appl. No.: 18/635,349

(22) Filed: Apr. 15, 2024

(51) Int. Cl. G16H 40/60; G16H 40/40

(52) U.S. Cl. CPC G16H 40/60; G16H 40/40

(57) ABSTRACT

A method for monitoring, analyzing, and adjusting performance of an individual medical device includes collecting, by the individual medical device, performance data of the individual medical device, identifying, by the individual medical device, the at least one variable using the collected performance data, analyzing, by the individual medical device, the at least one variable by comparing the at least one variable to a predetermined threshold, and if the at least one variable is not as expected, at least one of alerting, by the individual medical device, an operator of the individual medical device and adjusting, by the individual medical device, one or more operating parameters for the individual medical device.

### FIELD

The present disclosure relates to systems and methods for tracking information in individual medical devices (e.g., apheresis devices) to help identify issues with the individual medical device and to help update the individual medical device.

### BACKGROUND

This section provides background information related to the present disclosure which is not necessarily prior art.

There are two common methods for blood donation/collection. A first common method includes obtaining whole blood donation from a donor. Once the whole blood is obtained a centrifugal process may be used to separate blood components from the whole blood, for example, based on the density of different the blood component. The desired components can be manually, semi-automatically, or automatically moved to a collection container during and/or after application of the centrifugal forces. A second common method may be referred to as an apheresis collection, which requires a specialized machine. For example, the apheresis method may extract whole blood from a donor while the donor is connected to the specialized apheresis machine. The whole blood may then be centrifuged to collect only the desired blood component(s) (e.g., plasma) returning all other blood components to the donor during the same donation connection or cycle. The donor is connected to the apheresis machine during the separation and collection of the blood component. Unfortunately, the apheresis process can be lengthy and uncomfortable for some donors. Often, the donor must remain connected to the machine for an hour to obtain a blood component donation. Accordingly, making the donation procedure more efficient is an ongoing desire for apheresis collection sites.

### SUMMARY

This section provides a general summary of the disclosure and is not a comprehensive disclosure of its full scope or all of its features.

The present disclosure provides a method for monitoring, analyzing, and adjusting performance of an individual medical device.

In at least one example embodiment, the method may include analyzing, by the individual medical device, at least one variable and, if the at least one variable is not as expected, at least one of alerting, by the individual medical device, an operator of the individual medical device and adjusting, by the individual medical device, one or more operating parameters for the individual medical device.

In at least one example embodiment, the method may further include identifying the at least one variable. The at least one variable may be identified by an operator of the individual medical device.

In at least one example embodiment, the method may further include collecting, by the individual medical device, performance data of the individual medical device.

In at least one example embodiment, performance data of the individual medical device may be collected during the on-going cycle of the individual medical device and one or more previous cycles of the individual medical device.

In at least one example embodiment, the method may further include storing, by the individual medical device, at least a portion of the collected performance data.

In at least one example embodiment, the method may further include identifying, by the individual medical device, the at least one variable using the collected performance data.

In at least one example embodiment, the at least one variable may be identified, by the individual medical device, when the collected performance data exceeds a predetermined threshold.

In at least one example embodiment, the at least one variable may be identified, by the individual medical device, when an out of ordinary shift is experienced in the collected performance data. The out of ordinary shift may include instances where the collected performance data deviates by an amount greater than a determined standard deviation.

In at least one example embodiment, the standard deviation may be determined, by the individual medical device, using the collected performance data from an on-going cycle of the individual medical device.

In at least one example embodiment, the standard deviation may be determined, by the individual medical device, using the collected performance data from one or more previous cycles of the individual medical device.

In at least one example embodiment, the at least one variable may be analyzed after a selected time period.

In at least one example embodiment, the at least one variable may be analyzed when the at least one variable exceeds a predetermined threshold.

In at least one example embodiment, the at least one variable may be analyzed when an out of ordinary shift is experienced in the at least one variable. The out of ordinary shift may include instances where the at least one variable deviates by an amount greater than a determined standard deviation.

In at least one example embodiment, the analyzing may include comparing the at least one variable to a predetermined threshold value and if the at least one variable is greater than the predetermined threshold value, at least one of: the operator of the individual medical device is alerted; and one or more operating parameters are adjusted.

In at least one example embodiment, the method may further include collecting, by the individual medical device, performance data of the individual medical device, and the predetermined threshold value may be determined, by the individual medical device, using the performance data of the individual medical device.

In at least one example embodiment, the analyzing may include comparing the at least one variable to a predetermined threshold value and if the at least one variable is lower than the predetermined threshold value, at least one of: the operator of the individual medical device is alerted; and one or more operating parameters are adjusted.

In at least one example embodiment, the method may further include collecting, by the individual medical device, performance data of the individual medical device, and the predetermined threshold value may be determined, by the individual medical device, using the performance data of the individual medical device.

The present disclosure provides a method for monitoring, analyzing, and adjusting performance of an individual medical device.

In at least one example embodiment, the method includes collecting, by the individual medical device, performance data of the individual medical device, identifying, by the individual medical device, the at least one variable using the collected performance data, analyzing, by the individual medical device, the at least one variable by comparing the at least one variable to a predetermined threshold, and if the at least one variable is not as expected, at least one of alerting, by the individual medical device, an operator of the individual medical device and adjusting, by the individual medical device, one or more operating parameters for the individual medical device.

In at least one example embodiment, the performance data of the individual medical device may be collected during the on-going cycle of the individual medical device and one or more previous cycles of the individual medical device.

In at least one example embodiment, the at least one variable may be identified, by the individual medical device, when an out of ordinary shift is experienced in the collected performance data. The out of ordinary shift may include instances where the collected performance data deviates by an amount greater than a determined standard deviation.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a perspective view of an operating environment of an example apheresis system in accordance with at least one example embodiment of the present disclosure;

FIG. 2 is a perspective view of the example apheresis system illustrated in FIG. 1, where the access door is in a closed position in accordance with at least one example embodiment of the present disclosure;

FIG. 3 is a perspective view the example apheresis system illustrated in FIGS. 1-2, where the access door is in an open position revealing a centrifuge assembly in accordance with at least one example embodiment of the present disclosure;

FIG. 4 is a functional diagram of an example embodiment of the example apheresis system illustrated in FIGS. 1-3 in accordance with at least one example embodiment of the present disclosure;

FIG. 5 is a block diagram of an example electrical system of the example apheresis system illustrated in FIGS. 1-4 in accordance with at least one example embodiment of the present disclosure;

FIG. 6 is a further block diagram of the example electrical system of example apheresis system illustrated in FIGS. 1-5 in accordance with at least one example embodiment of the present disclosure;

FIG. 7 is a further block diagram of the example electrical system of example apheresis system illustrated in FIGS. 1-5 in accordance with at least one example embodiment of the present disclosure;

FIG. 8 is a flowchart illustrating an example method of using the apheresis system illustrated in FIGS. 1-7 in accordance with at least one example embodiment of the present disclosure;

FIG. 9 is a perspective view of the example apheresis system illustrated in FIGS. 1-8 further including a scanner in accordance with at least one example embodiment of the present disclosure;

FIG. 10 is a block diagram of an example computing system of the example apheresis system illustrated in FIGS. 1-9 in accordance with at least one example embodiment of the present disclosure;

FIG. 11 shows the example apheresis system illustrated in FIGS. 1-10 connected to a network in accordance with at least one example embodiment of the present disclosure;

FIG. 12 illustrates an example graphical user interface of the example apheresis system illustrated in FIGS. 1-10 in accordance with at least one example embodiment of the present disclosure;

FIG. 13 is a flowchart illustrating an example method for performing fleet management (including software updates and modifications) using a single apheresis system as an introduction point for a group of apheresis systems in accordance with at least one example embodiment of the present disclosure;

FIG. 14 is a flowchart illustrating another example method for performing fleet management (including software updates and modifications) using a single apheresis system as an introduction point for a group of apheresis systems in accordance with at least one example embodiment of the present disclosure;

FIG. 15 is a flowchart illustrating yet another example method for performing fleet management (including software updates and modifications) using a single apheresis system as an introduction point for a group of apheresis systems in accordance with at least one example embodiment of the present disclosure;

FIG. 16 is a flowchart illustrating an example method for tracking information in individual medical devices, where the tracked information can be used to help identify issues with the individual medical device and to help update the individual medical device, in accordance with at least one example embodiment of the present disclosure;

FIG. 17 is a flowchart illustrating another example method for tracking information in individual medical devices, where the tracked information can be used to help identify issues with the individual medical device and to help update the individual medical device, in accordance with at least one example embodiment of the present disclosure; and

FIG. 18 is a flowchart illustrating another example method for tracking information in individual medical devices, where the tracked information can be used to help identify issues with the individual medical device and to help update the individual medical device, in accordance with at least one example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

The present disclosure relates to methods of and means for collecting one or more blood components, like plasma, using a medical device or system, such as an apheresis device or system, like the apheresis devices or systems described in U.S. Pat. No. 11,090,425, titled METHODS AND SYSTEMS FOR HIGH-THROUGHOUT BLOOD COMPONENT COLLECTION and issued Aug. 17, 2021; U.S. application Ser. No. 18/117,035, titled SOFT CASSETTE WITH INTEGRATED FEATURES and filed Mar. 3, 2023; U.S. application Ser. No. 18/116,919, titled BLOOD COMPONENT COLLECTION BLADDER and filed Mar. 3, 2023; U.S. application Ser. No. 18/117,077, titled INTEGRATED CODE SCANNING SYSTEM AND APHERESIS DATA CONTROL METHOD and filed Mar. 3, 2023; U.S. application Ser. No. 18/117,044, titled METHODS AND SYSTEMS FOR THE CALIBRATION, MAINTENANCE, AND SERVICE OF APHERESIS SYSTEMS and filed Mar. 3, 2023; U.S. application Ser. No. 18/116,902, titled MOVING BLOOD COMPONENT COLLECTION LOOP HOLDER and filed Mar. 3, 2023; U.S. application Ser. No. 18/116,958, titled BOTTLE TRAY WITH MAGNETIC COUPLING AND LOAD CELL OVERLOAD PROTECTION and filed Mar. 3, 2023; U.S. application Ser. No. 18/116,988, titled COMMUNICATIONS AND OPERATION CONTROL OF APHERESIS SYSTEMS and filed Mar. 3, 2023; U.S. application Ser. No. 18/117,006, titled METHODS AND INTERFACES FOR PROVIDING DONATION PROCESS FEEDBACK and filed Mar. 3, 2023; U.S. application Ser. No. 18/117,007, titled MODULAR SERVICEABILITY SLEDS AND INTERCONNECTIONS and filed Mar. 3, 2023; U.S. application Ser. No. 18/116,908, titled COLLECTION BOTTLE WITH INTEGRATED CAP, HANDLE, AND SHIELD FEATURES and filed Mar. 3, 2023; U.S. application Ser. No. 18/116,992, titled METHODS FOR PROVIDING AUTOMATIC FLOW ADJUSTMENTS and filed Mar. 3, 2023; U.S. application Ser. No. 18/116,999, titled APHERESIS SYSTEM SAFETY FEATURES and filed Mar. 3, 2023; U.S. application Ser. No. 18/117,029, titled AUTOMATIC OPERATIONAL CONTROL BASED ON DETECTED ENVIRONMENTAL STATE and filed Mar. 3, 2023; U.S. application Ser. No. 18/116,954, titled FLEXURE-BASED TUBING STATE SENSOR and filed Mar. 3, 2023; U.S. application Ser. No. 18/117,035, titled SOFT CASSETTE WITH INTEGRATED FEATURES and filed Mar. 3, 2023; and U.S. application Ser. No. 18/117,073, titled BLOOD COMPONENT COLLECTION SET WITH INTEGRATED SAFETY FEATURES and filed Mar. 3, 2023, the entire disclosures of which are hereby incorporated by references.

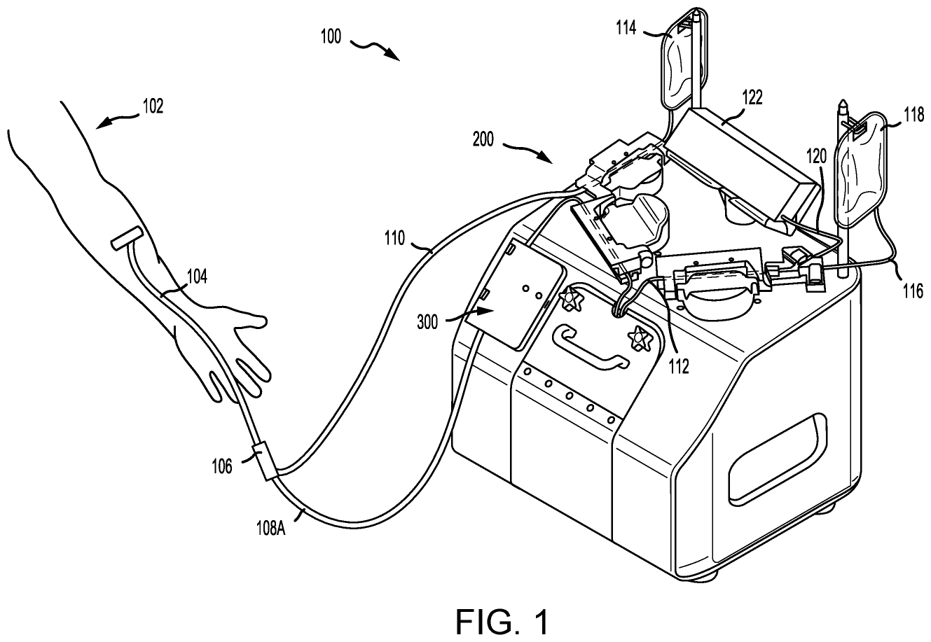

Apheresis systems generally include one or more connections configured to move whole blood and/or blood components to and from a blood component separation device housed within the apheresis system, where the blood component separation device is a centrifuge. FIG. 1 is a perspective view of an operating environment 100 of an apheresis system 200 in accordance with at least one example embodiment of the present disclosure. The operating environment 100 may include an apheresis system 200, a donor 102, and one or more connections (e.g., donor feed tubing 104, cassette inlet tubing 108A, anticoagulant (AC) tubing 110, etc.) running from the donor 102 to the apheresis system 200 and/or vice versa. The donor feed tubing 104 may be fluidly connected with at least one blood vessel, for example, a vein, of the donor 102 via venipuncture. For example, a cannula connected to an end of the donor feed tubing 104 may be inserted through the skin of the donor 102 and into a target site, or vein. This connection may provide an intravenous path for blood to flow from the donor 102 to the apheresis system 200 and/or for blood components to flow back to the donor 102. The whole blood supplied from the donor 102 may flow along the donor feed tubing 104 through a tubing connector 106 and along the cassette inlet tubing 108A into a soft cassette assembly 300. The soft cassette assembly 300 may include one or more fluid control paths and valves for selectively controlling the flow of blood to and/or from the donor 102. The apheresis system 200 may include an anticoagulant supply contained in an anticoagulant bag 114. The anticoagulant may be pumped at least through the anticoagulant tubing 110 and the tubing connector 106 preventing the coagulation of blood in the apheresis system 200.

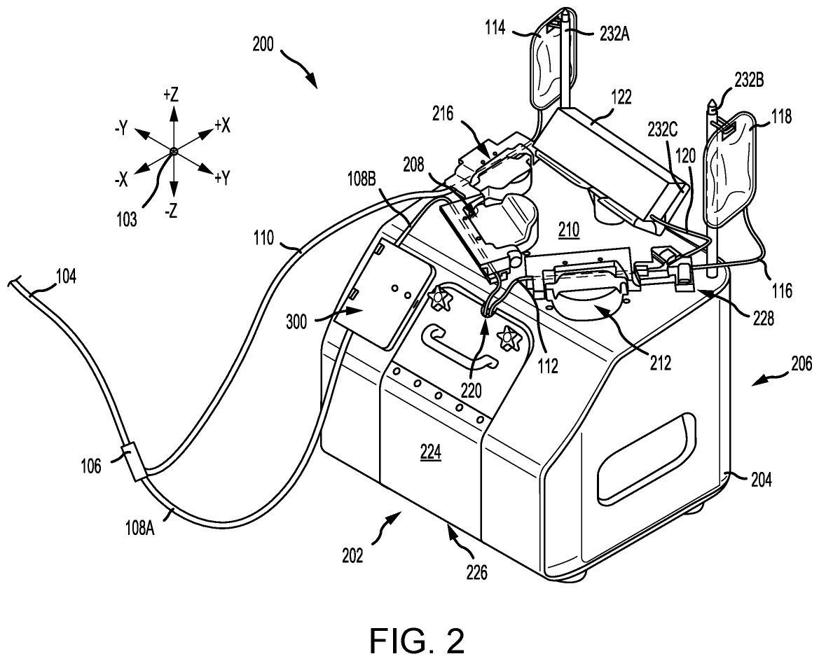

FIG. 2 is a perspective view of the apheresis system 200 described in FIG. 1. The apheresis system 200 may provide for a continuous whole blood separation process. In at least one example embodiment, the whole blood may be withdrawn from a donor 102 and substantially continuously provided to a blood component separation device of the apheresis system 200 where the blood may be separated into various components and at least one of these blood components may be collected from the apheresis system 200. In at least one example embodiment, one or more of the separated blood components may be either collected, for subsequent use, or returned to the donor 102. The blood may be withdrawn from the donor 102 and directed into a centrifuge of the apheresis system 200 through an opening 220 in an access panel 224 of the apheresis system 200. In at least one example embodiment, the tubing the donor feed tubing 104, the cassette inlet tubing 108A, inlet tubing 108B (also referred to herein as loop inlet tubing 108B), exit tubing 112 (also referred to herein as loop exit tubing 112), the saline tubing 116, and the plasma tubing 120, used in the extracorporeal tubing circuit may together define a closed, sterile, and disposable system (which may also be referred to as an exchange set and/or a blood component collection set and/or a fluid component collection set), which may be further described hereinafter.

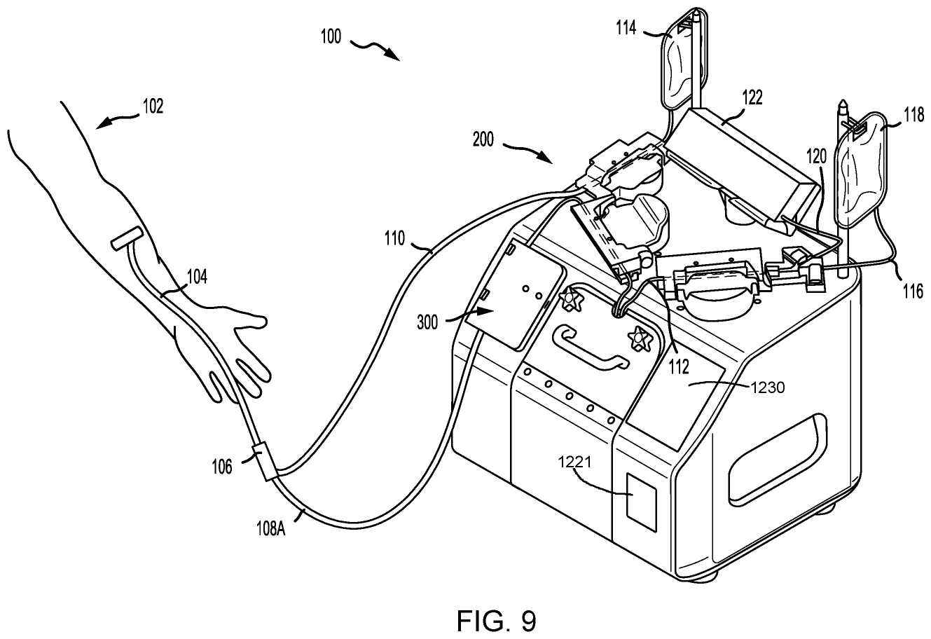

Operation of the various pumps, valves, and blood component separation device (e.g., centrifuge), may be controlled by one or more processors included in the apheresis system 200, and may advantageously comprise a plurality of embedded computer processors that are part of a computer system. The computer system may also include components that allow a user to interface with the computer system, including for example, memory and storage devices (RAM, ROM (e.g., CD-ROM, DVD), magnetic drives, optical drives, flash memory, etc.); communication/networking devices (e.g., wired such as modems/network cards, or wireless such as Wi-Fi); input devices such as keyboard(s), touch screen(s), camera(s), and/or microphone(s); and output device(s) such as display(s), and audio system(s), etc. It should be appreciated, that in at least one example embodiment, for example, as illustrated in FIG. 9, to assist the operator of the apheresis system 200 with various aspects of its operation, the blood component separation device (e.g., centrifuge), may include a graphical user interface with a display that includes an interactive touch screen.

The apheresis system 200 may include a housing 204 and/or structural frame, a cover 210, an access panel 224 disposed at a front 202 and/or rear 206 of the apheresis system 200, and one or more supports 232A-232C including hooks, rests, cradles, arms, protrusions, plates, and/or other support features for holding, cradling, and/or otherwise supporting a container or the anticoagulant bag 114, the saline bag 118, or the collection bottle 122. The housing 204 may include a machine frame (e.g., made of welded, bolted, and/or connected structural elements; extruded material; and/or beams) to which one or more panels, such as the cover 210, doors, subassemblies, and/or components are attached. In at least one example embodiment, at least one panel of the apheresis system 200 may include a mounting surface for the soft cassette assembly 300, one or more pumps such as a draw pump 208, a return pump 212, an anticoagulant pump 216, and/or a fluid valve control system 228 (e.g., plasma and saline valve control).

The access panel 224 may include one or more handles, locks, and a pivoting or hinged axis 226 (e.g., a door hinge, piano hinge, continuous hinge, cleanroom hinge, etc.). The access panel 224 may be selectively opened to provide access to an interior of the apheresis system 200, and more specifically, to a blood separation assembly, (e.g., centrifuge assembly). For example, the access panel 224 may provide access to load and/or unload the centrifuge with one or more components in the fluid component collection set. In at least one example embodiment, the inside of the apheresis system 200 may be separated into at least a centrifuge portion and a controls portion. For example, the centrifuge portion may include a cavity configured to receive the centrifuge, rotation motor, and associated hardware. This area may be physically separated from the controls portion via one or more walls of the cavity. In at least one example embodiment, access to the controls portion (e.g., configured to house or otherwise contain the motor controller, CPU or processor(s), electronics, and/or wiring) may be provided via a securely fastened panel of the housing 204 and/or panel separate from the access panel 224.

In at least one example embodiment, the apheresis system 200 may include a number of pumps, such as the draw pump 208, the return pump 212, and/or the anticoagulant pump 216, which can be configured to control the flow of fluid (e.g., blood and/or blood components, anticoagulant, and/or saline) through the apheresis system 200. As shown in FIG. 2, in at least one example embodiment, the draw pump 208, the return pump 212, and/or the AC pump 216 may be disposed at least partially on a top portion of the cover 210 of the apheresis system 200. In at least one example embodiment, the draw pump 208 may control blood flow to and/or from the donor 102 into the centrifuge of the apheresis system 200. For example, the draw pump 208 may engage with a portion of the inlet tubing 108B disposed between the soft cassette assembly 300 and the centrifuge of the apheresis system 200. In at least one example embodiment, the return pump 212 may be configured to control a flow of separated blood components (e.g., plasma) from the centrifuge to a collection bottle 122 and/or vice versa. Additionally, or alternatively, the return pump 212 may control a flow of saline (e.g., supplied from the saline bag 118) throughout the fluid component collection set and/or apheresis system 200. In at least one example embodiment, the anticoagulant pump 216 may engage with a portion of the anticoagulant tubing 110 to selectively control the flow of anticoagulant throughout the fluid component collection set of the apheresis system 200.

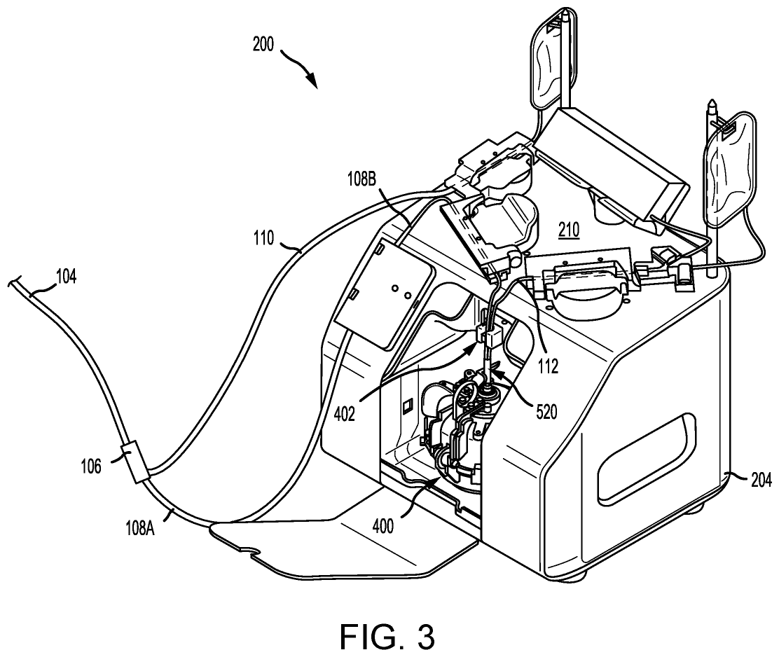

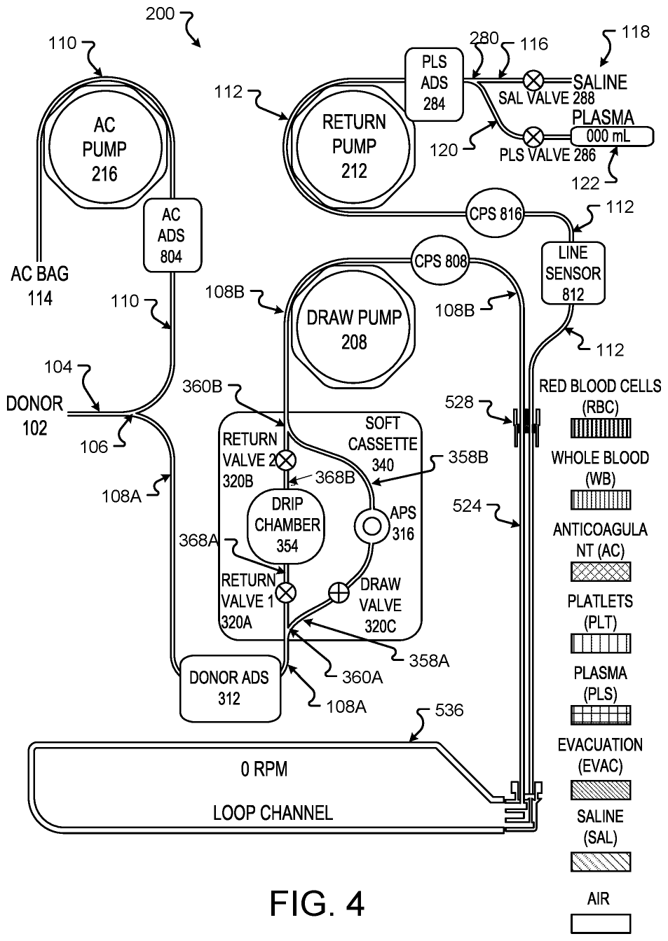

FIG. 3 illustrates an example centrifuge assembly 400 for use with or in the apheresis system 200 in accordance with at least one example embodiment of the present disclosure. For example, the centrifuge assembly 400 may be disposed in an interior space of the apheresis system 200. The interior space may be at least partially enclosed with one or more elements of the housing 204 and/or centrifuge chamber. Access to the interior space and the centrifuge assembly 400 may be provided via the access panel 224 disposed at the front 202 of the apheresis system 200. For example, in FIG. 4A, the access panel 224 is shown in an open position, opened along the hinged axis 226. The hinged axis 226 may correspond to a door hinge, continuous hinge, cleanroom hinge, and/or other panel hinges.

The centrifuge assembly 400 may be operatively mounted inside the apheresis system 200 such that the assembly 400 is capable of rotating relative to the housing 204 and/or other elements of the apheresis system 200. The centrifuge assembly 400 may be loaded with one or more portions of the fluid component collection set by routing tubing (e.g., the inlet tubing 108B and the exit tubing 112) into the interior space of the apheresis system 200 (e.g., via the opening 220 shown in FIG. 2), connecting a portion of a blood component collection loop 520 to the fixed loop connection 402 and inserting a blood component collection bladder into a filler 460. The fixed loop connection 402 maintains the inlet tubing 108B and the exit tubing 112 in a fixed position and may prevent twisting of the tubing 108B, 112 outside of the apheresis system 200. In at least one example embodiment, the blood component collection loop may be interconnected to the fixed loop connection 402 via one or more keyed features or positive location features.

FIG. 4 provides a functional diagram of the apheresis system 200 illustrated in FIGS. 1-3. FIG. 4 shows the components as previously introduced in a functional diagram to describe the operation of the system 200 for extracting plasma or other blood components from the whole blood of a donor 102 during an apheresis procedure or process. For example, as illustrated in FIG. 4, the anticoagulant pump 216 may be configured to pump fluid in the anticoagulant tubing 110 from the anticoagulant bag 114. In at least one example embodiment, the anticoagulant tubing 110 may also include an anticoagulant air detection sensor (ADS) 804 configured to detect air or fluid within the anticoagulant tubing 110. The anticoagulant tubing 110 may intersect with and be fluidly associated with the donor feed tubing 104 and also the cassette inlet tubing 108A at tubing connector 106. The tubing connector 106 may be any type of connection configured to connect the different tube 110, 104, and/or 108A.

The donor feed tubing 104 proceeds from the donor 102, where the donor 102 may be stuck with a lumen needle or other device, allowing whole blood to flow from the donor 102 into the apheresis system 200 and allowing blood components to flow back to the donor 102. The tubing 108A proceeds to the soft cassette 340. In at least one example embodiment, a donor air detection sensor (ADS) 312 can be placed on or in the tubing 108A. The donor air detection sensor may be configured to detect the presence of fluid and/or air within tubing 108A.

The soft cassette 340 may include a first cassette port 360A where the tube 108A breaks into first and second tubing sections. For example, the first cassette port 360A can function as, include, and/or be substantially proximate to a “Y” connector or section that separates the tubing 108A into the first bypass branch 358A and the first tubing section 368A. The two tubing sections and can reconnect at the second cassette port 360B, which like the first cassette port 360A, can function as, include, and/or be substantially proximate to a “Y” connector or section. In at least one example embodiment, the first tubing may be bisected by a fluid sensor 316 forming a first bypass branch 358A and a second bypass branch 358B. In at least one example embodiment, the first bypass branch 358A may include a first fluid control valve 320C. In at least one example embodiment, the second tubing may be bisected by the drip chamber 354 forming a first tubing section 368A and a second tubing section 368B. The drip chamber 354 may be configured to collect a volume of whole blood and/or high hematocrit blood (blood with a high percentage of red blood cells) depending on the operation of the system 200. In at least one example embodiment, the first tubing section 368A may include a second fluid control valve 320A, while the second tubing section 368B includes a third fluid control valve 320B. The fluid control valves 320A, 320B, 320C may be configured to isolate the different tubing portions 358A, 368A, 358B, 368B.

The inlet tubing 108B may be connected to the second cassette port 360B and may connect the soft cassette 340 to the flexible loop 524. In at least one example embodiment, the inlet tubing 108B may include a sensor 808, disposed on or in the tubing 108B before a system static loop connector 528 of the flexible loop 524. In at least one example embodiment, the inlet tubing 108B may include a pressure sensor (CPS) 808 disposed between the second cassette port 360B and the system static loop connector 528. In each instance, a draw pump 208 may be configured to pump fluid through the tubing 108B either away from the soft cassette 340 or towards the soft cassette 340.

Two or more different tubes may be connected to the flexible loop 524 via the system static loop connector 528 and may provide fluid to, or receive fluid from, a blood component collection bladder 536. In at least one example embodiment, an exit tubing 112 may be connected to the flexible loop 524 via the system static loop connector 528. In at least one example embodiment, the exit tubing 112 may include another line sensor 812 that is disposed thereon or therein and is configured to detect fluid, air, cellular concentration, color, and/or color change in the fluid moving from the flexible loop 524. In at least one example embodiment, a second pressure sensor or fluid sensor 816 may also be disposed in or on line 112. The sensor 816 may be configured to detect one or more of the presence or absence of fluid within the exit tubing 113, pressure within the exit tubing 112, and/or other characteristics of the fluid in exit tubing 112. In each instance, the exit tubing 112 may flow into a plasma air detection sensor 284 before a saline and plasma tubing y-connector 280 separates the exit tubing 112 into saline tubing 116 and plasma tubing 120. In at least one example embodiment, a return pump 212 may engage with the exit tubing 112 to cause fluid or air to flow through the exit tubing 112 from either the flexible loop 524 or a saline bag 118 and/or a collection bottle 122.

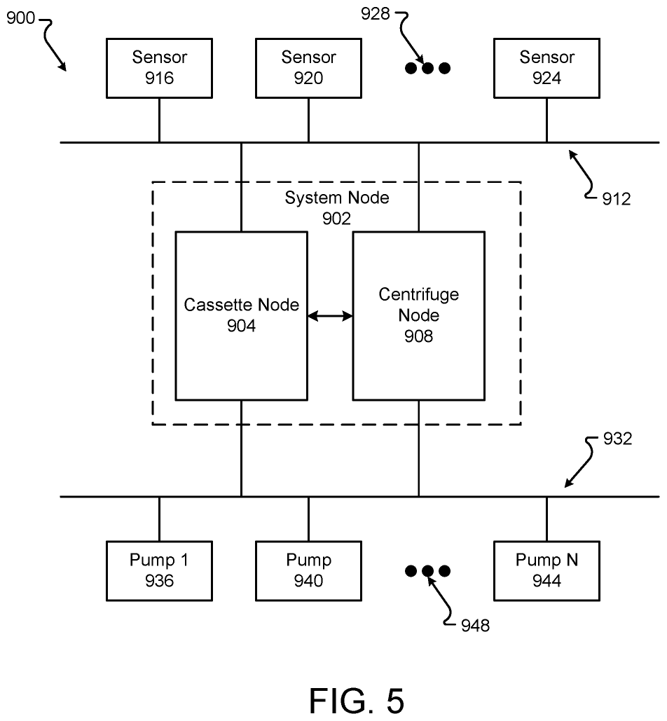

FIG. 5 illustrates an example electrical and control system 900 configured to control the functions of the apheresis system 200 illustrated in FIGS. 1-4. Generally, the control system 900 may include one or more nodes, which can include various hardware, firmware, and/or software configured to control and/or communicate with the mechanical, electromechanical, and electrical components of the apheresis system 200. Each node may be configured to control a different part of the apheresis system 200. For example, the control system 900 may include a cassette node 904 which may be a soft cassette assembly system may be configured to control and/or communicate with the components of a fluid component collection set (and/or the associated hardware or mechanical components interfacing with fluid component collection set or components thereof). The centrifuge node 908 which may be a centrifuge system may be configured to control and/or communicate with the centrifuge assembly 400 (and/or the associated hardware or mechanical components associated the with the centrifuge assembly 400 or components thereof). In at least one example embodiment, the cassette node 904 and centrifuge node 908 may be in communication with each other either wirelessly or through some other electrical or data connection. In at least one example embodiment, the cassette node 904 and centrifuge node 908 may separate portions of a single node or system 902. In at least one example embodiment, the cassette node 904 and centrifuge node 908 may have the same or different physical hardware that is used to operate and/or control different functions.

Each of the cassette node 904 and the centrifuge node 908 may be in communication with one or more sensors 916, 920, and/or 924. There may be more or fewer sensors than those shown in FIG. 5, as represented by ellipsis 928. Each of the cassette node 904 and/or the centrifuge node 908 can communicate directly to each sensor 916-924, or in other embodiments, may communicate with the several sensors 916-924 via a bus 912. The bus 912 may communicate by any type of communication protocol, such as universal serial bus (USB), a universal asynchronous receive/transmit (UART), or other types of bus systems or parallel communication connections. The sensors 916-924 can be any type of sensor that can communicate information, for example, about light, fluid, the presence of air, color, and/or pressure. Some of the sensors 916-924 may include sensors such as the air detection sensor 312, the fluid sensor 316, the anticoagulant air detection sensor 804, the pressure sensor 808, the line sensor 812, the second pressure sensor 816, and/or the air detection sensor 284.

Each of the cassette node 904 and the centrifuge node 908 may be in communication with one or more pump drives, pump motors, 936, 940, 944 (referred to collectively as “pumps”). There may be more or fewer pumps than are shown in FIG. 9, as represented by ellipsis 948. The cassette node 904 and/or the centrifuge node 908 may communicate with the pumps 936-944 through direct wired or wireless communication or through a bus 932. The bus 932 can be a control area network (CAN) bus, universal serial bus, or other type of bus architecture to communicate with the pumps 936-944. The pumps 936-944 can include, or be a part of, at least one of the draw pump 208, the return pump 212, and/or the anticoagulant pump 216.

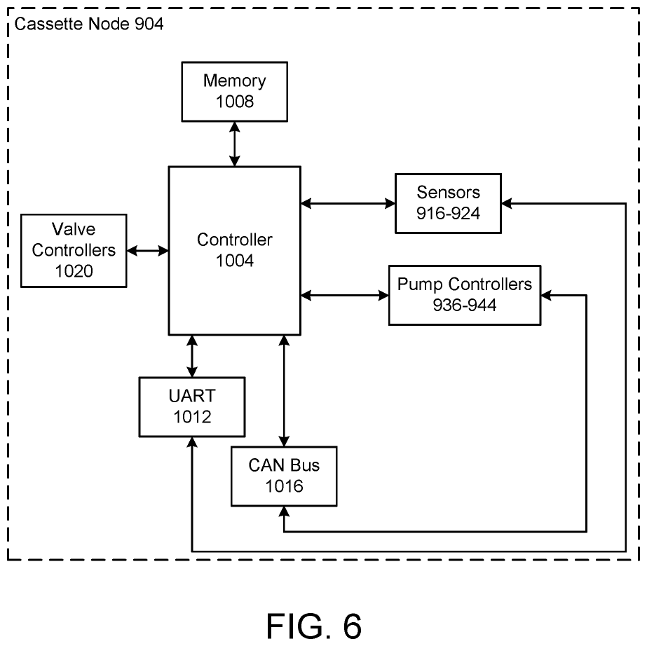

FIG. 6 illustrates an example cassette node 904. As illustrated, in at least one example embodiment, the cassette node 904 may include one or more of a controller 1004, a memory 1008, a valve controller 1020, a communication interface for a control area network bus 1016, a communication interface for a universal asynchronous receive/transmit 1012, and a communication interface for other types of buses. Although not illustrated, it should be recognized that the cassette node 904 can also include other hardware, firmware, and/or software.

The controller 1004, which may also be referred to as a processor, may be any type of microcontroller, microprocessor, Field Programmable Gate Array (FPGA), Application Specific Integrated Circuit (ASIC), etc. An example controller 1004 may be the NK10DN512VOK10 microcontroller, made and sold by N9P USA, Incorporated, which is a microcontroller unit with a 32-bit architecture. Other types of controllers, however, are possible. In each instance, the controller 1004 is configured to control other types of devices and/or to direct the functions of other types of devices, such as valves such as the first fluid control valve 320A, the second fluid control valve 320B, the draw fluid control valve 320C, the plasma flow control valve 286, the saline flow control valve 288, and/or the pumps 936-944. The controller 1004 may also be configured to communicate with various sensors 916-924 or other devices to receive and/or send information regarding the function of the apheresis system 200.

Other examples of the controller 1004, may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core i5-3570K 22 nm Ivy Bridge, the AMD® FX™ family of processors, AMD® FX-4300, FX-6300, and FX-8350 32 nm Vishera, AMD® Kaveri processors, ARM® Cortex™-M processors, ARM® Cortex-A and ARM926EJ-S™ processors, other industry-equivalent processors, and may perform computational functions using any known or future-developed standard, instruction set, libraries, and/or architecture.

The memory 1008 can be any type of memory including random access memory (RAM), read only memory (ROM), electrically erasable programmable ROM (EEPROM), a portable compact disc read-only memory (CD-ROM), an optical storage device, a magnetic storage device, any suitable combination of the foregoing, or other type of storage or memory device that stores and provides instructions to program and control the controller 1004. The memory 1008 may provide all types of software or firmware that programs the functions of the controller 1004.

The controller 1004 can communicate with one or more valve controllers 1020. Valves, such as such as the first fluid control valve 320A, the second fluid control valve 320B, the draw fluid control valve 320C, the plasma flow control valve 286, and/or the saline flow control valve 288, may be controlled by a valve controller 1020 and may be associated with a component of the system 200. The valve controller 1020 can provide the electrical signal, operational directive, or power to close or open any one of the valves, for example, the saline and plasma valve housing 276, the plasma flow control valve 286, the saline flow control valve 288, the first fluid control valve 320A, the second fluid control valve 320B, and/or the draw fluid control valve 320C.

The controller 1004 can also be connected to a bus 912, 932 through transceivers 1012, 1016 provided outside of the controller 1004 or integral to the controller 1004. The universal asynchronous receive/transmit transceiver 1012 may communicate with one or more of the sensors 916-924 or other devices. Likewise, the control area network bus transceiver 1016 can communicate with one or more of the pump controllers 936-944 or other devices. universal asynchronous receive/transmit transceivers 1012 and busses and control area network bus transceivers 1016 and busses are well known in the art and need not be explained further herein.

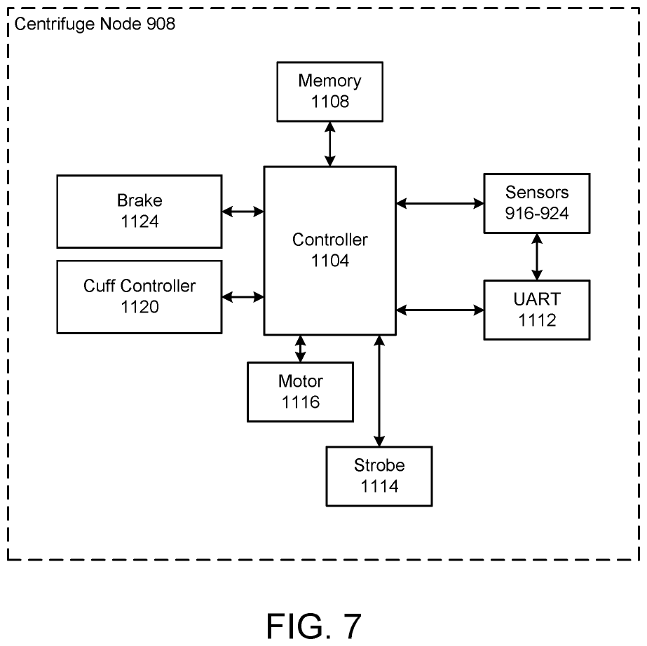

FIG. 7 illustrates an example centrifuge node 908. The centrifuge node can include the same or similar types of components as the cassette node 904. For example, in at least one example embodiment, as illustrated, the centrifuge node 908 may include a controller 1104 and/or a universal asynchronous receive/transmit transceiver 1112. Similar to the controller 1004, in at least one example embodiment, the controller 1104 may include any type of processor or microcontroller, including, for example, the NK10DN512VOK10 microcontroller unit with 32-bit architecture from N9P USA, Incorporated.

In at least one example embodiment, the controller 1104 may be configured to communicate with the sensors 916-924, for example, directly, through the universal asynchronous receive/transmit transceiver 1112 and/or through other busses or systems. In at least one example embodiment, the controller 1104 may be configured to communicate with a brake controller 1124. The brake controller 1124 may be configured to brake or slow and stop the centrifuge 400. In at least one example embodiment, the controller 1104 may be configured to communicate with a motor transceiver 1116. The motor transceiver 1116 may be configured to communicate with a motor power system or a motor controller that functions to spin up or rotate the centrifuge 400 and/or control the speed setting or other function of the centrifuge 400. In at least one example embodiment, the controller 1104 may be configured to communicate with a cuff controller 1120 that can change or set the pressure of a pressure cuff on a donor's 102 arm during the apheresis process. In at least one example embodiment, the controller 1104 may be configured to communicate a strobe light 1114 which can be any light that flashes at a periodicity in synchronicity with the rate of spin of the motor, such that an operator of the apheresis system 200 can see the operation of the filler 460. The controller 1104 may be configured to communicate with the strobe light 1114 so as to change the frequency of the flashing of the strobe light 1114 and/or the intensity of the strobe light 1114.

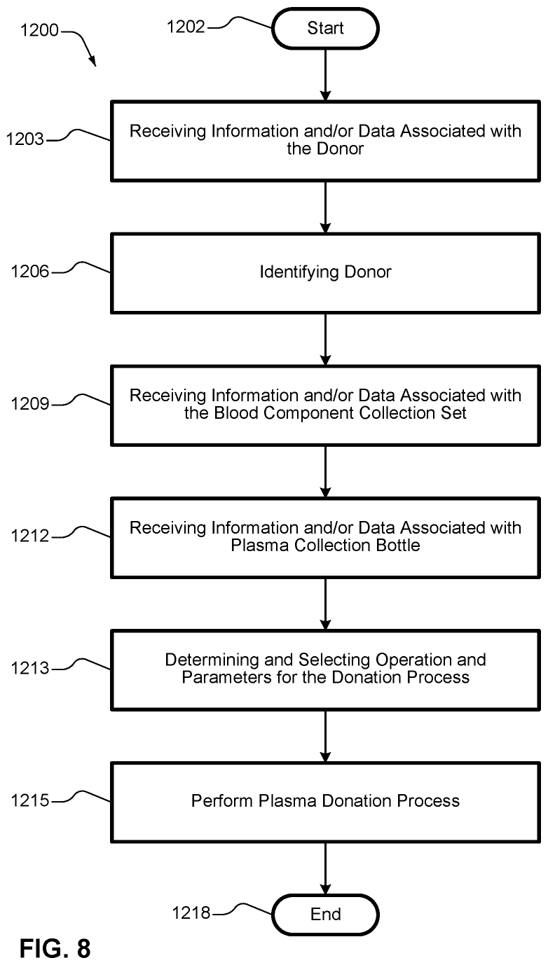

FIG. 8 is a flowchart illustrating an example data entry process 1200, for example, for initializing the apheresis system 200 illustrated in FIGS. 1-7. The data entry process 1200 may help to ensure, for example, that a target amount or volume of plasma and/or other blood component based on donor information is obtained and/or the quality of the plasma and/or other blood component. Also, in at least one example embodiment, that data entry process 1200, may allow for entry of other information, like bottle identification information and/or fluid component collection set such that the apheresis system 200 is capable of recording an indication as to which bottle and/or fluid component collection set was used for which donor 102. In at least one example embodiment, the data entry process 1200 may begin at 1202 where the apheresis system 200 is powered on to await a new donor 102.

In at least one example embodiment, as illustrated in FIG. 9, the apheresis system 200 may include an integrated identification reader (e.g., radio-frequency identification (RFID) reader, barcode reader, etc.) 1221 that is configured to read a code (e.g., radio-frequency identification tag, barcode, etc.) associated with a particular donor 102 and/or a code (e.g., radio-frequency identification tag, barcode, etc.) associated with a with a one or more components of a fluid component collection set and/or a code (e.g., radio-frequency identification tag, barcode, etc.) associated with a collection bottle to control and/or determine certain operation parameters of the apheresis system 200 in accordance with the information received by the identification reader. The information and/or data may include donor-specific information (e.g., biological information, including, for example, age, weight, height, hematocrit, hemoglobin, and/or donor history) and/or other information which may be relevant to the donation process (e.g., identity and/or relationship of one or more or more components of a fluid component collection set and/or collection bottle.

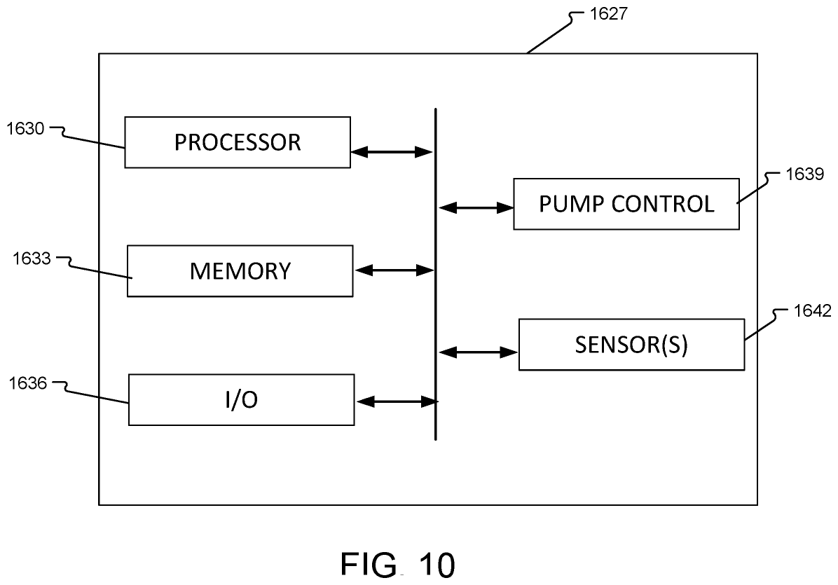

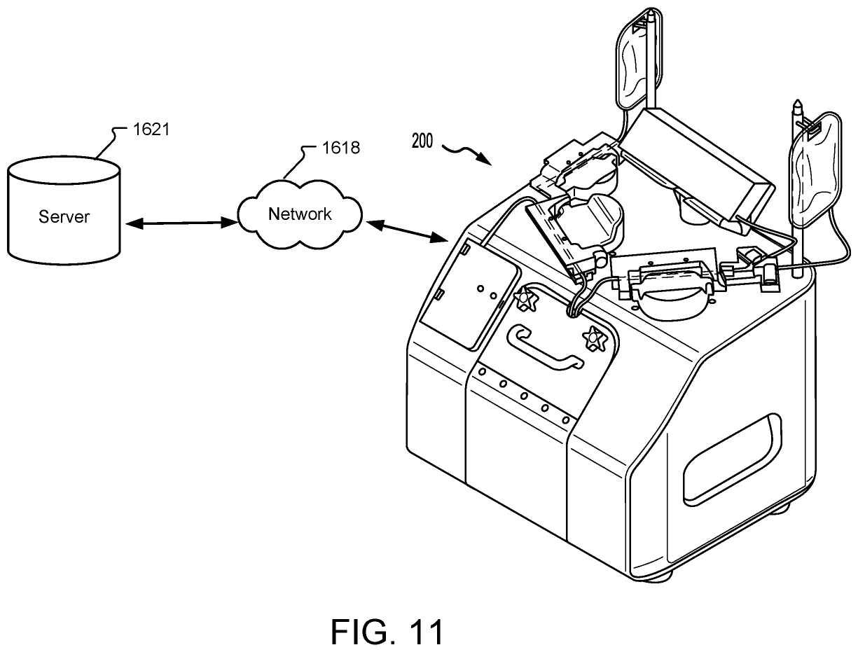

In at least one example embodiment, the apheresis system 200 may include one or more computer systems. For example, as illustrated in FIG. 10, the apheresis system 200 may include one or more computer systems 1627. The computer system(s) 1627 may include, for example, a processor 1630, memory 1633, input/output devices 1636, one or more pump control systems 1639, one or more sensors 1642, and/or other appropriate elements. The processor 1630 may be configured to execute software. The software may include firmware, applications, and/or operating systems which may manage execution of the apheresis system 200. The computer system(s) 1627 may be configured to detect the start 1202 of the apheresis system 200 and/or may be configured to transmit certain information and/or data to a server 1621 via a connection to a network 1618, as illustrated in FIG. 11. In this manner, the apheresis system 200 may communicate certain information and/or data (e.g., a data log, a firmware version identifier, and an error log) with a local system, such as a computer on location at a donation site, which may also be configured to communicate with the server 1621.

With renewed reference to FIG. 8, in at least one example embodiment, the data entry process 1200 may include receiving 1203 the donor-specific information and/or data and/or receiving 1209 information and/or data associated with the one or more components of a fluid component collection set and/or receiving 1212 information and/or data associated with the collection bottle. For example, the apheresis system 200 may be configured to scan a barcode, quick release code, or other type of image to receive donor-specific information, for example, from an identification card, and/or to received specific information about the one or more components of a fluid component collection set and/or collection bottle.

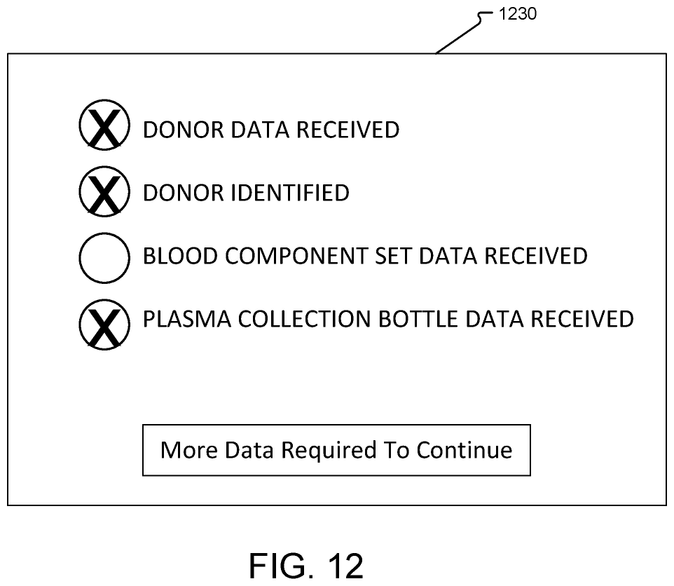

In at least one example embodiment, after receiving 1203 the donor-specific information and/or receiving 1209 the data associated with the one or more components of a fluid component collection set and/or receiving 1212 the data associated with the collection bottle, the apheresis system 200 may be configured to confirm receipt of the information and/data using a feedback system, such as a graphical user interface (GUI) 1230, as illustrated in FIG. 12. In this manner, a nurse, practitioner, or other user or operator of the apheresis system may be able to quickly ascertain whether the donor information and/or data has been properly inputted into the apheresis system 200. In at least one example embodiment, the feedback system may also, or alternatively, include a speaker that may be configured to provide audible feedback. For example, the one or more computer systems 1627 may use the graphical user interface (GUI) 1230 to guide the nurse, practitioner, or other user or operator of the apheresis system through one or more in-line sampling processes and/or to implement soft/hard enforcement of in-line sampling.

With renewed reference to FIG. 8, in at least one example embodiment, the method 1200 may further include, using the received donor-specific information and/or data to identify 1206 the donor 102. For example, in at least one example embodiment, the apheresis system 200 (for example, using the control system 900 and/or the computer system(s) 1627) may be configured to identify 1206, using the information and/or data received via the scanner 1221, whether the donor 102 is associated with any existing donor 102 identity in a database and/or whether the donor 102 is a new donor.

The method 1200 may also include receiving 1209 information and/or data associated with a fluid component collection set to be used during the donation process. In at least one example embodiment, the fluid component collection set may include a soft cassette assembly, like the soft cassette assembly 300. In at least one example embodiment, the information and/or data associated with the fluid component collection set may be received 1209 by the apheresis system 200 via a barcode, quick release code, radio-frequency identification chip, and/or other type of scannable object that may be associated with the fluid component collection set. In at least one example embodiment, each fluid component collection set may be affixed with a label or sticker that includes a distinct barcode, quick release code, radio-frequency identification chip, and/or other type of scannable object. By scanning the label or sticker on the fluid component collection set, the apheresis system 200 may be enabled to record into memory (and in at least one embodiment, shared with the server 1621) which fluid component collection set is being used for the current donation process. In this manner, the apheresis system 200 may be enabled to associate donor with a selected fluid component collection set and/or one or more components thereof. The data associated with the fluid component collection set may include, for example, a date of manufacture, an identity of manufacturer, and/or various other information that may be useful for data processing purposes after the donation is complete. In at least one example embodiment, data associated with the fluid component collection set received 1209 through scanning may be used to determine a type of fluid component collection set. The type of fluid component collection set may be used by the apheresis system 200 to adjust one or more settings such as flow rate or other information during the donation process.

In at least one example embodiment, after scanning the fluid component collection set, an operator of the apheresis system 200 may receive confirmation of the receipt of the information from the fluid component collection set. For example, a graphical user interface 1230, as illustrated in FIG. 12, may display an indication as to whether the information and/or data from a fluid component collection set has been received. In at least one example embodiment, instead of or in addition to displaying through a graphical user interface 1230, the apheresis system 200 may play an audible sound through one or more speakers or display lights of various colors to indicate the data has been received.

With renewed reference to FIG. 8, in at least one example embodiment, the method 1200 includes receiving 1212 data associated with a collection bottle. For example, in at least one example embodiment, to initialize the apheresis system 200 for a new donor, a collection bottle may be required. After donation, the collection bottle may be filled with the donated plasma (or other fluid). For data tracking purposes, the collection bottle may be required to be associated with the donor making it may be necessary for an identity of the collection bottle to be recorded. As such, an operator of the apheresis system 200 may be enabled to scan a label, sticker, or other object associated with (e.g., printed on or sticked to) the collection bottle using the apheresis system 200. As with the previously detailed steps, upon receiving 1212 the information and/or data from a plasma collection bottle, the apheresis system 200 may acknowledge receipt of the data via a graphical user interface 1230, speaker, white, and/or other feedback system.

In at least one example embodiment, the method 1200 may use the received data and/or information (including the donor-specific information and/or data and/or the blood component collect set information and/or data and/or the collection bottle information and/or data) to determine and/or selected 1213 one or more operating parameters of the donation process. For example, in at least one example embodiment, the apheresis system 200 may be configured (for example, via the control system 900 and/or the computer system(s) 1627) to calculate the amount or volume of plasma (or other fluid) the donor 102 can provide or donate using the information and/or data received, such as further detailed in Atty. Docket No. 18955-000224-US-PSI, titled METHODS AND SYSTEMS FOR DETERMINING DONOR SPECIFIC AMOUNTS FOR COLLECTION USING TARGETED CHANGES IN HEMATOCRIT and listing John Pittinger and Thomas J. Felt as inventors, as filed Apr. 29, 2023 and assigned U.S. App. No. 63/462,636, the entire contents of which are herein incorporated by reference.

The method includes performing 1215 the donation or collection process based on information received and/or detailed as detailed above. In at least one example embodiment, the method 1200 may end at 1218, at which point the donation process may continue with the extraction of fluids from the donor being completed. Any information and/or data received through the above discussed steps may be recorded into memory and/or shared with one or more computer systems 1627. For example, a database entry may be created for the particular donation, including information such as an amount or volume of plasma extracted from the donor, a current weight of the donor, a time and/or date of the donation, and/or other information and/or data.

Software, including firmware, applications, operating systems, and other programmable features of the apheresis system 200, may be updated from time to time to ensure apheresis system 200 is operating as expected and/or to add new features and/or functionality. In at least one example embodiment, the apheresis system 200 may include and/or interface with (for example, via a network connection) an application that, among other things, performs fleet management, including receiving and analyzing device data and also allowing software updates and changes to be applied to the fleet of apheresis systems or one or more groups defining the fleet, where the fleet of apheresis systems or the one or more groups defining the fleet include apheresis systems like apheresis system 200. Each of the one or more groups may include two or more apheresis systems.

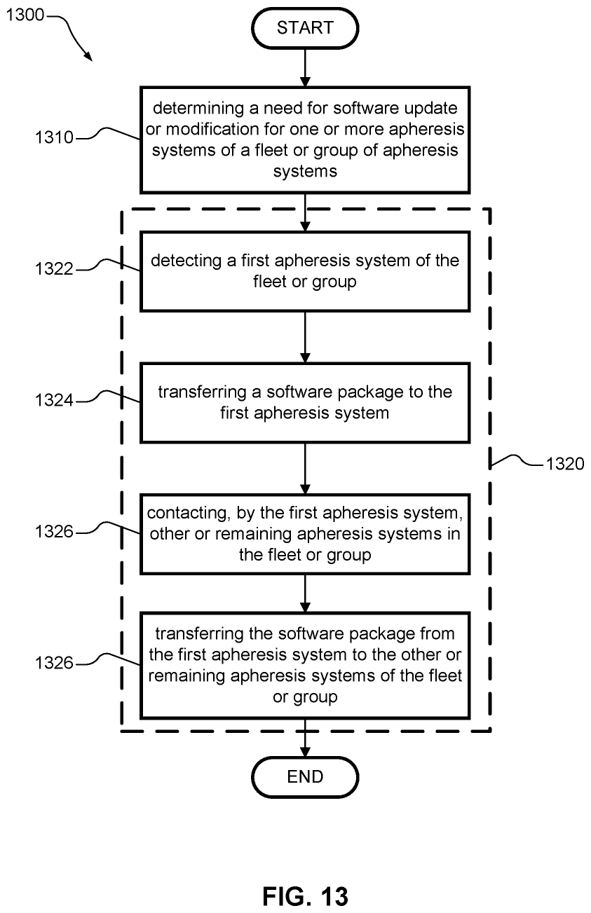

As illustrated in FIG. 13, in at least one example embodiment, a method 1300 for using the application, for example, by control or controller (like control system 900), may include determining 1310 if the software (including, for example, firmware, applications, operating systems, and other programmable features) of any of the apheresis systems in a fleet of apheresis systems and/or any of the apheresis systems in one or more groups of apheresis systems in the fleet of apheresis systems should be or needs to be updated or modified. For example, each apheresis system in the fleet or in the one or more groups may communicate information (for example, during startup) about the software, including firmware version, error logs encountered, and/or the like to a remote system server (like server 1621) via a communications network (like communications network 1618). In at least one example embodiment, software system implemented by the apheresis system(s) may be configured to generate and/or compile device logs (referred to, for example, as D-logs) and to send the D-logs to a cloud storage location that is in communication with the remote system server.

The remote system server may be configured to determine whether the software of any of the apheresis systems in the fleet or any of the apheresis systems in the one or more groups needs to be updated or modified. The remote system server may be configured to force a software update or modification automatically and/or give a user an option to select to update or modify the software. The user may select to update or modify immediately or select a certain future day and time. In at least one example embodiment, an external device may be connected to (e.g., in communication with) one or more of the apheresis system and/or the remote system server. The external device may include one or more computer systems that are configured to communicate the software update or modification to at least one of the apheresis systems in the fleet or to at least one of the apheresis systems in the group of apheresis systems or to at least one of the apheresis systems in a first group of apheresis systems and to at least one of the apheresis systems in a second group of apheresis systems. For example, when it is determined 1310 that the software of any of the apheresis systems in the fleet or any of the apheresis systems in the one or more groups should be or needs to be updated or modified, the method 1300 may include initiating 1320 a software push from the external device and/or remote system server. Delaying the software push until a need is identified by the external device and/or remote system server and/or user can help to limit impacts on a network for a center or location that houses and/or uses the apheresis systems.

In at least one example embodiment, the initiating 1320 of the software push may include detecting 1322 one apheresis system (i.e., a first apheresis system) of the fleet or one apheresis system (i.e., a first apheresis system) of the group of apheresis system or one apheresis system (i.e., a first apheresis system) of a first group of the one or more groups of apheresis systems and one apheresis system (i.e., a second, first apheresis system) of a second group of the one or more groups of apheresis systems; transferring 1324 a software package to the first apheresis system (and/or the second, first apheresis system); contacting 1326 by the first apheresis system (and/or the second, first apheresis system) other or remaining or peer apheresis systems in the fleet or the one or more group; and transferring 1326 the software package from the first apheresis system (and/or the second, first apheresis system) to the other apheresis systems in the fleet or the one or more groups. In at least one example embodiment, the contacting 1326 by the first apheresis system (and/or the second, first apheresis system) of the other or remaining or peer apheresis systems in the fleet or the one or more groups at the center or location that houses and/or uses the apheresis systems may include the first apheresis system (and/or the second, first apheresis system) announcing the availability of the software package to the other or remaining or peer apheresis systems in the fleet or the one or more groups and establishing a state to which the other or remaining or peer apheresis systems must sync. In at least one example embodiment, the contacting 1326 by the first apheresis system (and/or the second, first apheresis system) of the other or remaining or peer apheresis systems in the fleet or the one or more groups at the center or location that houses and/or uses the apheresis systems may include the first apheresis system (and/or the second, first apheresis system) providing the software package to one or more of the other or remaining or peer apheresis systems and, as synchronization progresses, still other or remaining or peer apheresis systems receiving and/or pulling the software package (e.g., blocks of data) from the one or more of the other or remaining or peer apheresis systems that initially received the software package from the first apheresis system (and/or the second, first apheresis system) in a web-like fashion. In each instance, peer-to-peer transfer 1326 (via a chain event or a group event or a web-like event) can help to minimize traffic between a location or center's network and the external source or location hosting the software package. After a software package is transferred from the external source or location to the first apheresis system (and/or the second, first apheresis system) on site at the location or center that houses and/or uses the apheresis systems, propagating the package to the peer devices in a group may use the location or center's internal local area network.

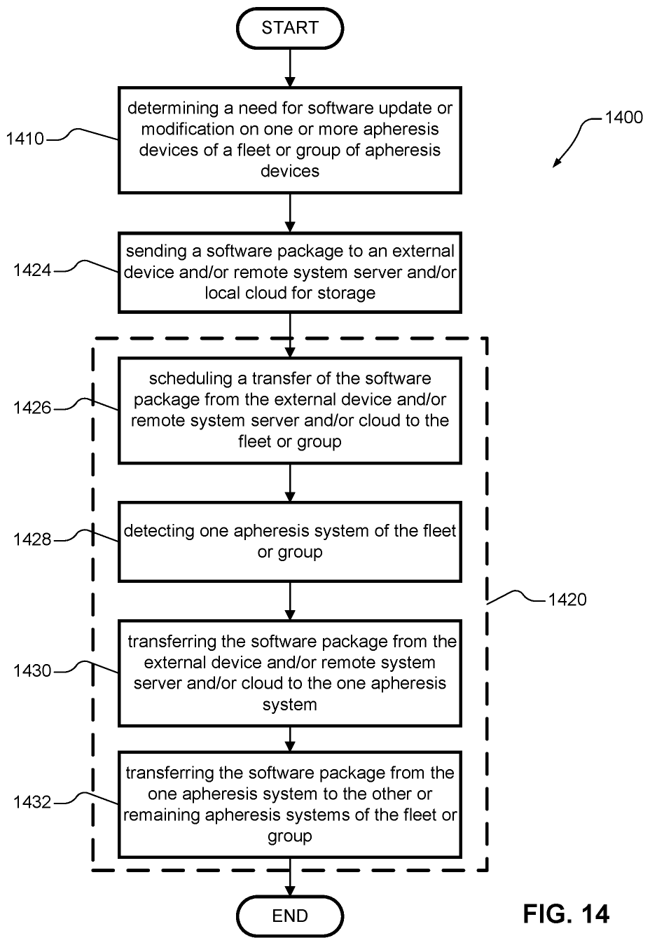

FIG. 14 is a flowchart illustrating another example method 1400 for performing fleet management (including software updates and/or modifications and/or maintenance) using a single apheresis system as an introduction point for a fleet of apheresis systems or one or more groups of apheresis systems within the fleet of apheresis systems. Like the method 1300, the method 1400 may include determining 1410 if the software (including, for example, firmware, applications, operating systems, and other programmable features) of any of the apheresis systems in the fleet or of any of the apheresis systems in the one or more groups of apheresis systems in the fleet of apheresis systems should be or needs to be updated or modified and when it is determined 1410 that the software of any of the apheresis systems in the fleet or that any of the apheresis systems in the one or more groups of apheresis systems should be or needs to be updated or modified, the method 1400 may include initiating 1420 a software push from the external device and/or remote system server and/or local cloud to the fleet or the one or more group of apheresis systems. In at least one example embodiment, the method 1400 may include sending or transferring 1424 a software package to the external device and/or the remote system server and/or the local cloud. Although the sending or transferring 1424 of the software package to the external device and/or the remote system server and/or the local cloud is illustrated as following the determining 1410, it should be appreciated that in at least one example embodiment, the sending or transferring 1424 may occur prior to the determining 1410.

In at least one example embodiment, the initiating 1420 of the software push may include scheduling 1426 a transfer of the software package from the external device and/or the remote server and/or the local cloud to the fleet or the one or more group of apheresis systems; detecting 1428 one apheresis system of the fleet of apheresis systems (i.e., a first apheresis system) or one apheresis system of the one or more groups of apheresis systems or a first apheresis system of a first group of the one or more groups of apheresis systems and a second, first apheresis system of a second group of the one or more groups of apheresis systems; transferring 1430 the software package to the first apheresis system (and/or the second, first apheresis system); and transferring 1432 the software package from the first apheresis system (and/or the second, first apheresis system) to the other apheresis systems.

In at least one example embodiment, the scheduling 1426 may be determined by the user or operator or customer or administrator using the fleet of apheresis systems or the one or more groups of apheresis systems at the location or center that houses and/or uses the apheresis systems. The scheduling 1426 may include setting a time window for the transfer of the software package from the external device and/or the remote server and/or the local cloud to the fleet of apheresis systems or the one or more groups of apheresis systems. In at least one example embodiment, the detecting 1428 may be considered automatic because the detection occurs as previously scheduled.

As in the instance of method 1300, in at least one example embodiment, the transferring 1432 of method 1400, may include the first apheresis system (and/or the second, first apheresis system) announcing the availability of the software package to the other or remaining or peer apheresis systems in the fleet of apheresis systems or the one or more groups of apheresis systems and establishing a state to which the other or remaining or peer apheresis systems must sync. In at least one example embodiment, the transferring 1432 of method 1400, may include the first apheresis system (and/or the second, first apheresis system) providing the software package to one or more of the other or remaining or peer apheresis systems and, as synchronization progresses, still other or remaining or peer apheresis systems receiving and/or pulling the software package (e.g., blocks of data) from the one or more of the other or remaining or peer apheresis systems that initially received the software package from the first apheresis system (and/or the second, first apheresis system) in a web-like fashion.

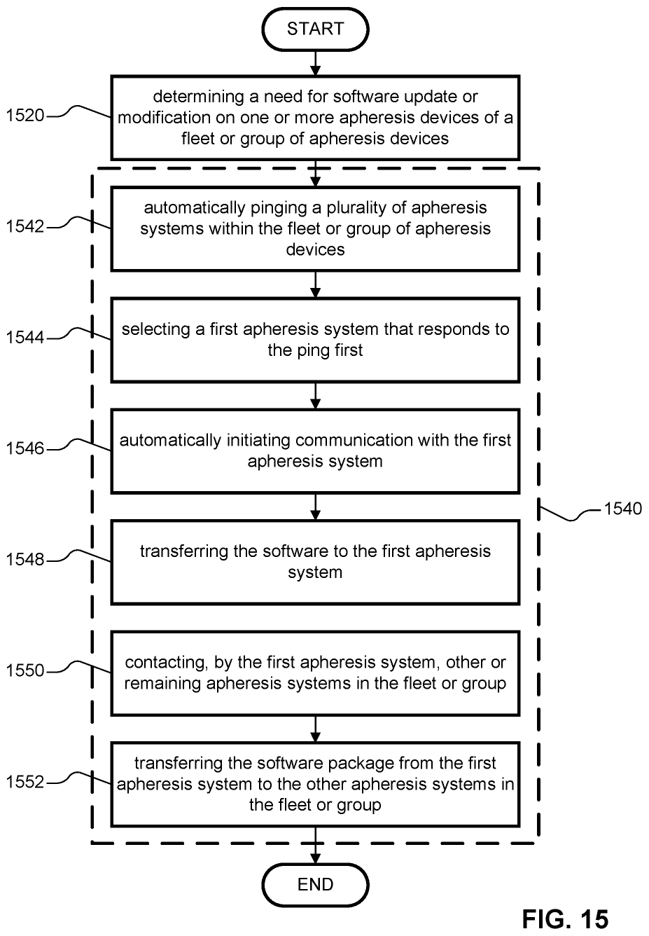

FIG. 15 is a flowchart illustrating yet another example method 1500 for performing fleet management (including software updates and/or modifications and/or maintenance) using a single apheresis system as an introduction point for a fleet or group of apheresis systems. The method 1500 may include, like methods 1300 and 1400, determining 1520 if the software (including, for example, firmware, applications, operating systems, and other programmable features) of any of the apheresis systems in the fleet or group should be or needs to be updated or modified. When it is determined 1520 that the software of any of the apheresis systems in the fleet or group should be or needs to be updated or modify, the method 1500 may include transferring 1540 the software package from the cloud to apheresis systems in the fleet or group.

In at least one example embodiment, transferring 1540 the software package may include pinging 1542 a plurality of apheresis systems in the fleet or in the one or more groups; selecting 1544 an apheresis system that responds to the ping first (i.e., first apheresis system); initiating 1546 communication with the first apheresis system at a predetermined time (e.g., a user-configured window of time); transferring 1548 the software package from to the first apheresis system; contacting 1550 by the first apheresis system other apheresis systems in the fleet or the one or more groups; and transferring 1552 the software package from the first apheresis system to the other apheresis systems.

As in the instance of method 1300 and also method 1400, in at least one example embodiment, the transferring 1552 of method 1500, may include the first apheresis system announcing the availability of the software package to the other or remaining or peer apheresis systems in the fleet or the one or more groups and establishing a state to which the other or remaining or peer apheresis systems must sync. In at least one example embodiment, the transferring 1552 of method 1500, may include the first apheresis system providing the software package to one or more of the other or remaining or peer apheresis systems and, as synchronization progresses, still other or remaining or peer apheresis systems receiving and/or pulling the software package (e.g., blocks of data) from the one or more of the other or remaining or peer apheresis systems that initially received the software package from the first apheresis system in a web-like fashion.

Although methods 1300, 1400, 1500 are illustrated as distinct methods, it should be appreciated that, in various example embodiments, the method steps of any of 1300, 1400, 1500 may be combined together in one or more configurations.

In various aspects, it may be advantageous, in addition to, or alternatively to, the fleet or group update, to be able to collect or gather or record and process data on the level of an individual apheresis system (like the apheresis system 200), for example, allowing the individual apheresis system to monitor normal range of the particular sensors specific to the individual system and/or allowing the individual apheresis system to determine if the number of particular identified events is normal or abnormal and/or allowing the individual apheresis system to be an active determinative of the health of the individual apheresis system setting individual goals and targets for servicing and calibration. Computer systems (e.g., one or more computer systems 1627) embedded (or forming part of) the apheresis system may be provided the ability to record, track, and process data. For example, the computer system may be programmed to calculate results and determine certain actions automatically (using, for example, machine learning or other advance algorithms), where the data can span multiple data logs across multiple procedures and time and the results are specific to the individual apheresis system and more, which can be more accurate than limits or thresholds set for a population.

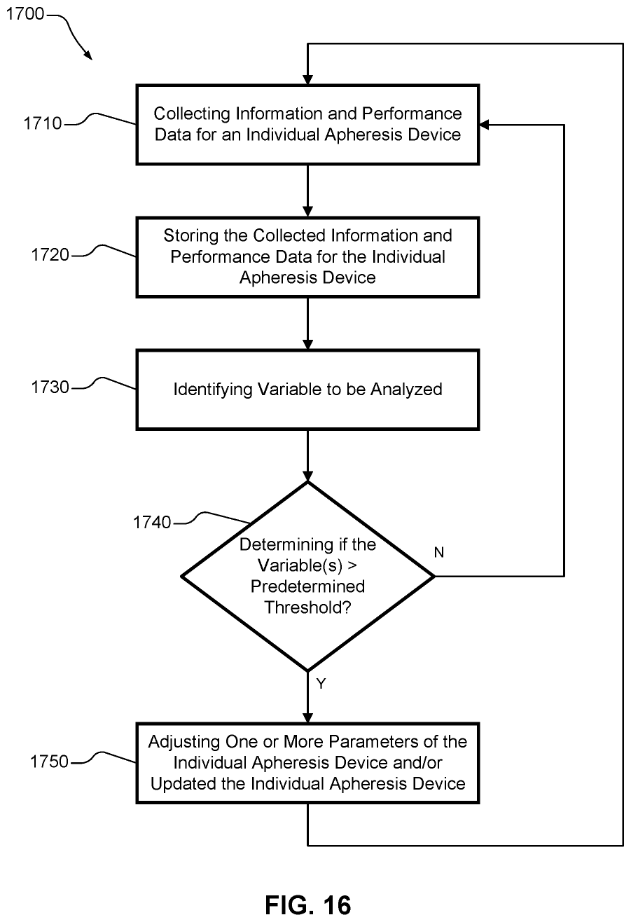

As illustrated in FIG. 16, in at least one example embodiment, a method 1700 for tracking information in an individual apheresis device (like the apheresis system 200) to help identify issues with the individual apheresis device and to help update the individual apheresis device may include, for example, by control or controller (like control system 900 and/or the one or more computer systems 1627), collecting 1710 information and performance data for the individual apheresis device and storing or logging 1720 at least a portion of the collected information and performance data, for example, in a memory (like memory 1008 and/or memory 1633). At determined intervals (for example, after a predetermined amount of data is collected or measured and/or after a predetermined amount of time and/or after any of the collected information or performance data exceeds one or more predetermined thresholds and/or after an out of ordinary shift in any of the collected or measured information or performance data), the method 1700 may include identifying 1730 at least one variable to be considered or analyzed from the collected or measured information and performance data and comparing 1740 the identified variable or variables to a predetermined threshold or limit. The variable or variables to be considered or analyzed may be those, for example, that an out of ordinary shift has been identified. An out of ordinary shift is when the collected or measured data deviates, for example by an amount greater than the determined standard deviation, from like data during the on-going cycle and/or previous cycles.

The predetermined threshold may be an individual value or range determined for the individual apheresis device. For example, although not illustrated, it should be appreciated that, in various example embodiments, the method 1700 may include determining the predetermined threshold. The predetermined threshold may be determined using, and dependent on, data collected or measured regarding the performance of the individual apheresis device during the on-going cycle and/or previous cycles. In each instance, if the variable or variables are lower than the predetermined threshold (or alternatively, in other embodiments, greater than the predetermined threshold), the method 1700 will return to 1710. If the variable or variables are greater than the predetermined threshold (or alternatively, in other embodiments, lower than the predetermined threshold), the method 1700 will proceed to 1750, where one or more parameters of the apheresis device are adjusted and/or the apheresis device updated. Although not illustrated, it should be appreciated that, in various example embodiments, the method 1700 may include announcing or presenting or identifying the adjustment and/or the update to and for the nurse, practitioner, or other user or operator of the apheresis system, for example, using the graphical user interface (GUI) 1230. Once the one or more parameters of the apheresis device are adjusted and/or the apheresis device updated, the method 1700 may return to 1710 to continue collection of information and performance data.

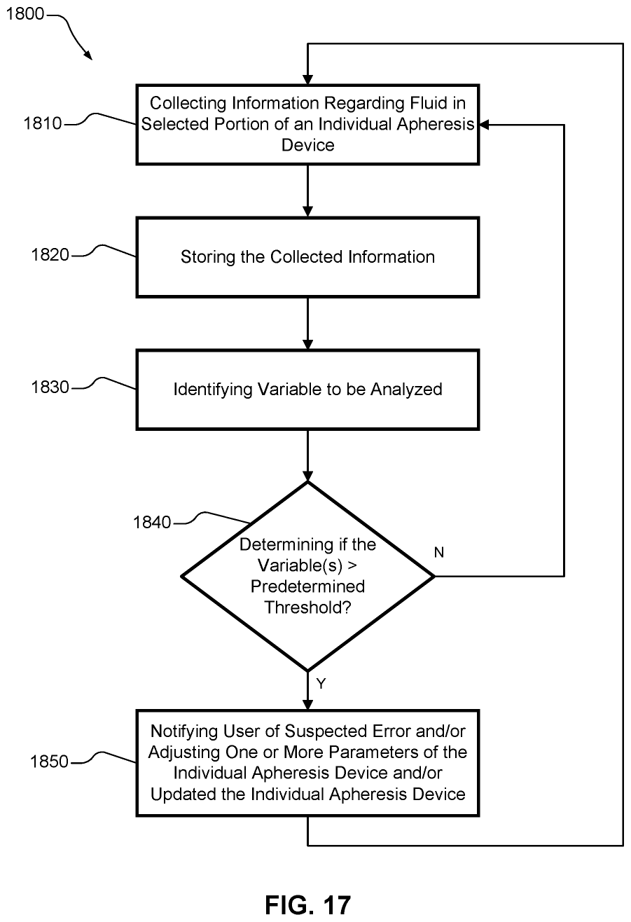

FIG. 17 is a flowchart illustrating an example method 1800 for tracking information in an individual apheresis device (like the apheresis system 200) to help identify issues with the individual apheresis device and to help update the individual apheresis device may include, for example, using a sensor, collecting 1810 information regarding the presence of fluid in a selected portion of the apheresis device and storing or logging 1820 at least a portion of the collected information, for example, in a memory (like memory 1008 and/or memory 1633). The method 1800 may include identifying 1830 at least one variable to be considered or analyzed from the collected information. For example, when fluid is expected and not detected, the method 1800 may identify 1830 the absence as the variable to be considered or analyzed. The method 1800 will then compare the absence to a predetermined threshold or limit.

If the absences are lower than the predetermined threshold (or alternatively, in other embodiments, greater than the predetermined threshold), the method 1800 will return to 1810. If the variable or variables are greater than the predetermined threshold (or alternatively, in other embodiments, lower than the predetermined threshold), the method 1800 will proceed to 1850, where the nurse, practitioner, or other user or operator of the apheresis system is notified of the error and/or one or more parameters of the apheresis device are adjusted and/or the apheresis device updated. For example, if fluid is absence in three of six occurrences, the method 1800 will proceed with 1850 where the nurse, practitioner, or other user or operator of the apheresis system is notified of the error and/or one or more parameters of the apheresis device are adjusted and/or the apheresis device updated.

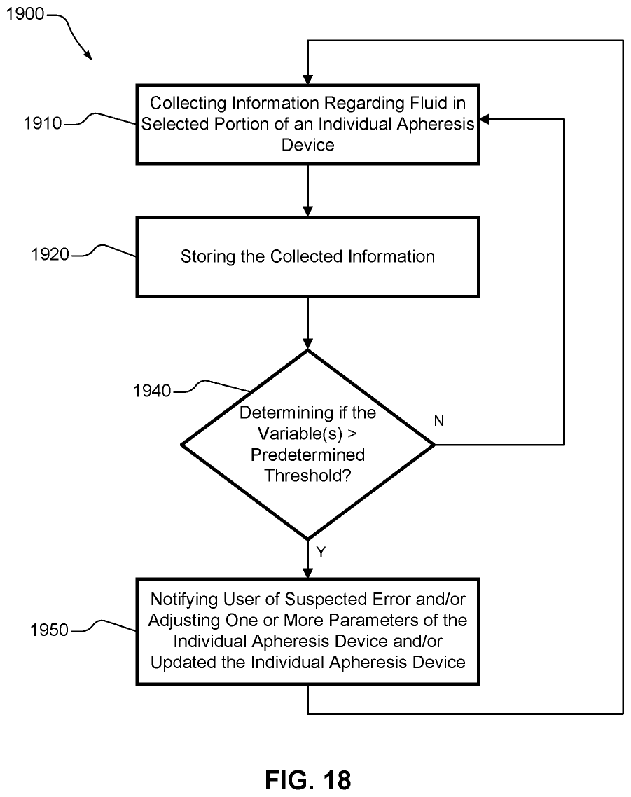

FIG. 18 is a flowchart illustrating an example method 1800 for tracking information in an individual apheresis device (like the apheresis system 200) to help identify issues with the individual apheresis device and to help update the individual apheresis device may include, for example, using a sensor, collecting 1810 information regarding calibration values of gain and offset for a sensor requiring calibration and storing or logging 1820 at least a portion of the collected information, for example, in a memory (like memory 1008 and/or memory 1633). The method 1800 may include collecting 1810 the information regarding calibration values of gain and offset for a sensor requiring calibration and storing 1820 at least a portion of the collected information for at least 10 calibration cycles, for example.

The method 1800 may include comparing 1940 any of the calibration cycles to a predetermined threshold. For example, if a new gain or offset value is attempted that is greater than to about 10% different from the previous range of values, the method 1900 may proceed to 1850, where the nurse, practitioner, or other user or operator of the apheresis system is notified of the error and/or one or more parameters of the apheresis device are adjusted and/or the apheresis device updated. For example, the calibration cycle or cycles may be rejected forcing the nurse, practitioner, or other user or operator of the apheresis system to repair and/or recalibrate. If a new gain or offset valve is attempted that is less than about 10% different from the previous range of values, the method 1900 may return to 1810.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for monitoring, analyzing, and adjusting performance of an individual medical device, the method comprising:
analyzing, by the individual medical device, at least one variable; and
if the at least one variable is not as expected, at least one of alerting, by the individual medical device, an operator of the individual medical device and adjusting, by the individual medical device, one or more operating parameters for the individual medical device.

2. The method of claim 1, wherein the method further includes:
identifying the at least one variable, the at least one variable being identified by an operator of the individual medical device.

3. The method of claim 1, wherein the method further includes:
collecting, by the individual medical device, performance data of the individual medical device.

4. The method of claim 3, wherein the performance data of the individual medical device is collected during the on-going cycle of the individual medical device and one or more previous cycles of the individual medical device.

5. The method of claim 3, wherein the method further includes:
storing, by the individual medical device, at least a portion of the collected performance data.

6. The method of claim 3, wherein the method further includes:
identifying, by the individual medical device, the at least one variable using the collected performance data.

7. The method of claim 6, wherein the at least one variable is identified, by the individual medical device, when the collected performance data exceeds a predetermined threshold.

8. The method of claim 6, wherein the at least one variable is identified, by the individual medical device, when an out of ordinary shift is experienced in the collected performance data, the out of ordinary shift including instances where the collected performance data deviates by an amount greater than a determined standard deviation.

9. The method of claim 8, wherein the standard deviation is determined, by the individual medical device, using the collected performance data from an on-going cycle of the individual medical device.

10. The method of claim 8, wherein the standard deviation is determined, by the individual medical device, using the collected performance data from one or more previous cycles of the individual medical device.

11. The method of claim 1, wherein the at least one variable is analyzed after a selected time period.

12. The method of claim 1, wherein the at least one variable is analyzed when the at least one variable exceeds a predetermined threshold.

13. The method of claim 1, wherein the at least one variable is analyzed when an out of ordinary shift is experienced in the at least one variable, the out of ordinary shift including instances where the at least one variable deviates by an amount greater than a determined standard deviation.

14. The method of claim 1, wherein the analyzing includes:
comparing the at least one variable to a predetermined threshold value and if the at least one variable is greater than the predetermined threshold value at least one of: the operator of the individual medical device is alerted; and one or more operating parameters are adjusted.

15. The method of claim 14, wherein
the method further includes collecting, by the individual medical device, performance data of the individual medical device; and
the predetermined threshold value is determined, by the individual medical device, using the performance data of the individual medical device.

16. The method of claim 1, wherein the analyzing includes:
comparing the at least one variable to a predetermined threshold value and if the at least one variable is lower than the predetermined threshold value at least one of: the operator of the individual medical device is alerted; and one or more operating parameters are adjusted.

17. The method of claim 16, wherein
the method further includes collecting, by the individual medical device, performance data of the individual medical device; and
the predetermined threshold value is determined, by the individual medical device, using the performance data of the individual medical device.

18. A method for monitoring, analyzing, and adjusting performance of an individual medical device, the method comprising:
collecting, by the individual medical device, performance data of the individual medical device;
identifying, by the individual medical device, the at least one variable using the collected performance data;
analyzing, by the individual medical device, the at least one variable by comparing the at least one variable to a predetermined threshold; and
if the at least one variable is not as expected, at least one of alerting, by the individual medical device, an operator of the individual medical device and adjusting, by the individual medical device, one or more operating parameters for the individual medical device.

19. The method of claim 18, wherein the performance data of the individual medical device is collected during the on-going cycle of the individual medical device and one or more previous cycles of the individual medical device.

20. The method of claim 18, wherein the at least one variable is identified, by the individual medical device, when an out of ordinary shift is experienced in the collected performance data, the out of ordinary shift including instances where the collected performance data deviates by an amount greater than a determined standard deviation.
